(19) United States
(12) Patent Application Publication
(10) Pub. No.: US 2023/0146680 A1
(43) Pub. Date: May 11, 2023

(54) METHOD FOR PRODUCING ENHANCED ANTI-INFLAMMATORY / ANTI-CATABOLIC AGENTS FROM AUTOLOGOUS PHYSIOLOGICAL FLUID

(71) Applicant: Antnor Limited, Toronto (CA)

(72) Inventors: Anthony Galea, Toronto (CA); Irina Brokhman, Vaughan (CA)

(21) Appl. No.: 17/906,385

(22) PCT Filed: Mar. 16, 2021
(86) PCT No.: PCT/CA2021/050351

§ 371 Date: Sep. 15, 2022

(30) Foreign Application Priority Data
Mar. 17, 2020 (CA) 3076046

(51) Int. Cl. A61K 35/19; A61K 35/14; A61K 35/16; A61K 38/20; A61K 38/55; A61K 47/12; A61P 19/02; A61P 29/00

(52) U.S. Cl. CPC A61K 35/19; A61K 35/14; A61K 35/16; A61K 38/2006; A61K 38/55; A61K 47/12; A61P 19/02; A61P 29/00

(57) ABSTRACT

A method of producing an autologous anti-inflammatory/anti-catabolic autologous composition useful in the treatment of a mammal suffering from damaged and/or injured connective tissues, chronic tendinosis, chronic muscle tears, chronic degenerative joint conditions, and/or skin inflammatory disorders is provided. The method comprising the following steps: delivering a blood collected from the mammal to a tube; storing the blood in presence of sodium citrate at a temperature of from about 20° C. to about 40° C. for at least about 3.5 hours; centrifuging the blood to separate the blood into a supernatant component and a cellular fraction; and collecting the supernatant component.

### TECHNICAL FIELD

The application is directed generally to medicine, and more particularly to methods and compositions useful, in among other things, the treatment of damaged and/or injured connective tissues including chronic tendinosis, chronic muscle tears (tendinitis), cartilage tears, chronic degenerative joint conditions such as osteoarthritis as well as chronic inflammatory skin diseases including, atopic dermatitis, chronic wounds and cosmetics.

### BACKGROUND

Osteoarthritis (“OA”) is a degenerative joint disease characterized by cartilage damage and synovial inflammation. Changes to a molecular inflammatory cascade lead to a destruction of cartilage macromolecules and irreversible morphological changes. IL-1, Tumor Necrosis Factor-alpha (TNFα), IL-6,8 and metalloproteinases are predominant catabolic and pro-inflammatory molecules that have a major role in the pathogenesis of osteoarthritis. These cytokines are produced by activated synoviocytes, mononuclear cells or by articular cartilage itself and their catabolic effect can be successfully blocked by inhibitory cytokines such as IL-4, 10, 13 and IL-1ra.

Similar inflammatory and catabolic pathways are involved in the pathogenesis of chronic tendonitis and chronic muscle tear healing failure. Tendon cells are subjected to continuous damage by producing increased levels of IL-1, 6, metalloproteinases (MMPs) and other catabolic molecules. Pro-inflammatory cytokines IL-1 and TNFα are involved in the pathogenesis of chronic myositis as well. Atopic dermatitis (eczema) is considered to be the most common relapsing inflammatory skin conditions. Chronic wound (including diabetic wound) is a wound that does not heal within three months due to poor circulation, neuropathy, immune disorders and complications of systemic illnesses, age, and repeated trauma. All of these conditions are characterized by disturbing cell signaling via cytokines and lost extracellular matrix (ECM) that forms the largest component of the dermal skin layer. Targeting special inflammatory and catabolic molecular pathways can have a beneficial therapeutic effect for inflammatory pathologies. This effect could be achieved by using therapeutically active proteins. Presently, the pharmaceutical industry employs high cost molecular genetic technologies for recombinant protein production such as insulin, interferons, blood clotting factors, etc. However, these methods of recombinant protein generation include the expression of human genes in a bacterial cell. The patterns of post-translation protein modification including glycosylation may be different than those naturally occurring in humans. This may result in instability of the product in the human environment, decreasing of biological function or immune response provocation. Additionally, the cost of the final recombinant product is extremely high.

U.S. Pat. No. 6,713,246 to Reinecke et al discloses that in order to prepare an anti-inflammatory/anti-catabolic composition, blood is incubated at body temperature for 24 hours in order for a sufficient amount of the anti-inflammatory/anti-catabolic factor IL-1ra to be produced in the incubated blood for therapeutic purposes. Previous studies show that in healthy individuals, an incubation time of blood of about 24 hours is required to produce a sufficient level of IL-1ra and other anti-inflammatory/anti-catabolic factors to provide a measurable therapeutic benefit.

There is a need for a method for treating damaged and/or injured connective tissues, chronic tendinosis, chronic muscle tears and/or chronic degenerative joint conditions such as osteoarthritis, and skin inflammatory disorders and for cosmetic applications where a therapeutically useful anti-inflammatory component/anti-catabolic component can be produced with a shorter incubation or storage time for the blood sample.

### SUMMARY OF THE DISCLOSURE

Described is an anti-inflammatory/anti-catabolic composition useful for treating damaged and/or injured connective tissues, chronic tendinosis, chronic muscle tears and/or chronic degenerative joint conditions such as osteoarthritis, and skin inflammatory disorders. Also described is a method for making the anti-inflammatory/anti-catabolic composition. The anti-inflammatory/anti-catabolic composition is produced by collecting the blood of individuals, mixing the blood with sodium citrate to form an admixture, and incubating or storing the admixture of blood and sodium citrate for a time period of at least about 3.5 hours at a temperature of from about 20° C. to about 40° C. The anti-inflammatory/anti-catabolic composition may also be combined with a regenerative composition that includes autologous platelet rich plasma (PRP) for treating damaged and/or injured connective tissues, chronic tendinosis, chronic muscle tears and/or chronic degenerative joint conditions such as osteoarthritis, and skin inflammatory disorders and for cosmetic applications. Where the anti-inflammatory/anti-catabolic composition is combined with a regenerative composition that includes autologous platelet rich plasma (PRP), the resulting composition is an autologous composition that is useful in the treatment of a mammal suffering from damaged and/or injured connective tissues, chronic tendinosis, chronic muscle tears, chronic degenerative joint conditions, and/or skin inflammatory disorders and for cosmetic applications. Where the anti-inflammatory/anti-catabolic composition is combined with the regenerative composition that includes autologous platelet rich plasma (PRP), the anti-inflammatory/anti-catabolic composition is an anti-inflammatory/anti-catabolic component of the resulting autologous composition whereas the regenerative composition is a regenerative component of the resulting autologous composition.

Where the blood of a human or other mammal is mixed with sodium citrate prior to incubation, the anti-inflammatory/anti-catabolic composition comprises an increased level IL-1ra after at least about 3.5 hours of the incubation of the blood mixed with sodium citrate at a temperature of from about 20° C. to about 40° C. In addition, the anti-inflammatory/anti-catabolic composition preferably comprises an increased and/or therapeutically effective level of tissue inhibitors of metalloproteinases (TIMPs) after at least about 3.5 hours of the incubation of the admixture at a temperature of from about 20° C. to about 40° C.

According to an aspect of the present disclosure, there is provided a method of producing an autologous anti-inflammatory/anti-catabolic autologous composition useful in the treatment of a mammal suffering from damaged and/or injured connective tissues, chronic tendinosis, chronic muscle tears, chronic degenerative joint conditions, and/or skin inflammatory disorders, the method comprising the following steps:
admixing blood from the mammal with a quantity of sodium citrate to form an admixture; incubating the admixture at a temperature of from about 20° C. to about 40° C. for at least about 3.5 hours to about 12 hours;
centrifuging the incubated admixture to separate the blood into a supernatant component and a cellular fraction; and
collecting the supernatant component.

According to another aspect, there is provided method of treating a mammal suffering from damaged and/or injured connective tissues, chronic tendinosis, chronic muscle tears and/or chronic degenerative joint conditions and skin inflammatory disorders with an autologous anti-inflammatory/anti-catabolic composition, the method comprising the following steps:
admixing blood from the mammal with a quantity of sodium citrate to form an admixture; incubating the admixture at a temperature of from about 20° C. to about 40° C. for at least about 3.5 hours to about 12 hours;
centrifuging the incubated admixture to separate the blood into a supernatant component and a cellular fraction;
collecting the supernatant component to provide the autologous anti-inflammatory/anti-catabolic composition; and
administering the autologous anti-inflammatory/anti-catabolic composition to the mammal.

According to another aspect, there is provided a method of producing an autologous composition useful in the treatment of a mammal suffering from damaged and/or injured connective tissues, chronic tendinosis, chronic muscle tears, chronic degenerative joint conditions, and/or skin inflammatory disorders, the method comprising the following steps:
preparing an anti-inflammatory/anti-catabolic component of the autologous composition comprising IL-1ra and TIMPs, said step of preparing the anti-inflammatory/anti-catabolic component comprising the following steps:
admixing blood from the mammal with a quantity of sodium citrate to form an admixture; incubating the admixture at a temperature of from about 20° C. to about 40° C. for at least about 3.5 hours to about 12 hours;
centrifuging the incubated admixture to separate the blood into a supernatant component and a cellular fraction;
collecting the supernatant component of the anti-inflammatory/anti-catabolic component;
preparing a regenerative component of the autologous composition comprising the following steps:
mixing blood from the mammal with a quantity of an anticoagulant which is preferably an about 4% by weight sodium citrate solution;
centrifuging the blood to separate a platelet rich plasma component therefrom; collecting the platelet rich plasma component; and
mixing the supernatant component of the anti-inflammatory/anti-catabolic component with the platelet rich plasma component to provide the autologous composition.

According to yet another aspect, there is provided a method of treating a mammal suffering from damaged and/or injured connective tissues, chronic tendinosis, chronic muscle tears and/or chronic degenerative joint conditions and skin inflammatory disorders, the method comprising the following steps:
preparing an anti-inflammatory/anti-catabolic component an autologous composition comprising IL-1ra and TIMPs, said step of preparing the anti-inflammatory/anti-catabolic component comprising the following steps:
admixing blood from the mammal with a quantity of sodium citrate to form an admixture; incubating the admixture at a temperature of from about 20° C. to about 40° C. for at least about 3.5 hours to about 12 hours;
centrifuging the incubated admixture to separate the blood into a supernatant component and a cellular fraction; and
collecting the supernatant component of the anti-inflammatory/ anti-catabolic component; preparing a regenerative component of the autologous composition comprising the following steps:
mixing blood from the mammal with a quantity of an anticoagulant which is preferably an about 4% by weight sodium citrate solution;
centrifuging the blood to separate a platelet rich plasma component therefrom; collecting the platelet rich plasma component;
mixing the supernatant component with the platelet rich plasma component to provide the autologous composition; and
administering the autologous composition to the mammal.

### DESCRIPTION OF THE DRAWINGS

FIG. 1 is a plot of IL-1ra concentration in pg/ml versus time showing a comparison of the level of IL-1ra antagonist protein in the human serum samples of patients having osteoarthritis at different time points.

FIG. 2 is a plot of TIMPs concentration in pg/ml versus time showing a comparison of the level of TIMP 1 and TIMP 2 in the human serum samples of patients having osteoarthritis at different time points.

FIG. 3 is plots i) showing a statistical analysis of Visual Analog Pain Scale (VAS) for a first patient tested; ii) showing point values according to the WOMAC index for level of pain for the first patient tested; iii) showing point values according to the WOMAC index for level of stiffness for the first patient tested; and iv) showing point values according to the WOMAC index for levels of daily activity capabilities for the first patient tested. Values are provided for a baseline and 1 month post injections.

FIG. 4 is plots i) showing a statistical analysis of Visual Analog Pain Scale (VAS) for a second patient tested; ii) showing point values according to the WOMAC index for level of pain for the second patient tested; iii) showing point values according to the WOMAC index for level of stiffness for the second patient tested; and iv) showing point values according to the WOMAC index for levels of daily activity capabilities for the second patient tested. Values are provided for a baseline and 1 month post injections.

FIG. 5 is plots i) showing a statistical analysis of Visual Analog Pain Scale (VAS) for a third patient tested; ii) showing point values according to the WOMAC index for level of pain for the third patient tested; iii) showing point values according to the WOMAC index for level of stiffness for the third patient tested; and iv) showing point values according to the WOMAC index for levels of daily activity capabilities for the third patient tested. Values are provided for a baseline and 1 month post injections.

FIG. 6 is plots i) showing a statistical analysis of Visual Analog Pain Scale (VAS) for a fourth patient tested; ii) showing point values according to the WOMAC index for level of pain for the fourth patient tested; iii) showing point values according to the WOMAC index for level of stiffness for the fourth patient tested; and iv) showing point values according to the WOMAC index for levels of daily activity capabilities for the fourth patient tested. Values are provided for a baseline and 1 month post injections.

FIG. 7 is a plot of IL-1ra concentration in pg/ml versus time showing a comparison of the level of IL-1ra antagonist protein in the human serum samples at different time points of 12 subjects tested.

FIG. 8 is a plot of MMP9 concentration in pg/ml versus time showing a comparison of the level of MMP9 in the human serum samples at different time points of the 12 subjects tested.

FIG. 9 is a plot of TNF-alpha concentration in pg/ml versus time showing a comparison of the level of TNF-alpha in the human serum samples at different time points of the 12 subjects tested.

FIG. 10 is a plot of IL-1b concentration in pg/ml versus time showing a comparison of the level of IL-1b in the human serum samples at different time points of the 12 subjects tested.

FIG. 11a is a plot showing a statistical analysis of Visual Analog Pain Scale (VAS) among 22 patients tested. Values are provided for a baseline and 1 month post injections.

FIG. 11b is a plot showing point values according to the WOMAC index for average levels of pain among 22 patients tested. Values are provided for a baseline and 1 month post injections.

FIG. 11c is a plot showing point values according to the WOMAC index for average levels of stiffness among 22 patients tested. Values are provided for a baseline and 1 month post injections.

FIG. 11d is a plot showing point values according to the WOMAC index for average levels of daily activity capabilities among 22 patients tested. Values are provided for a baseline and 1 month post injections.

### DETAILED DESCRIPTION

The disclosure relates to a method for producing an autologous anti-inflammatory/anti-catabolic composition that produces IL-1ra, TIMP 1 and TIMP 2 in sufficient quantities for therapeutic use when stored at room temperature for at least about 3.5 hours to about 6 hours or more at a temperature of about 20° C. to about 40° C.

The disclosure also relates to a method for producing an autologous composition comprising the autologous anti-inflammatory/anti-catabolic composition in combination with a regenerative autologous platelet-rich plasma (PRP) composition with serum enriched by bioactive proteins having a anti-inflammatory/anti-catabolic, proliferative, tissue remodeling and regenerative effects. With respect to the autologous composition referred to herein, the autologous anti-inflammatory/anti-catabolic composition is an anti-inflammatory/anti-catabolic component of the autologous composition whereas the regenerative autologous platelet-rich plasma (PRP) composition is a regenerative component of the autologous composition.

Such a composition typically includes the following therapeutically active proteins: IL-1ra, IL-4, IL-10, IL-13, PDGF, TGF-β and VEGF.

IL-1ra is secreted by monocytes, adipocytes and epithelial cells. It is known that therapeutically effective concentrations of this protein are achieved by incubating human monocytes from healthy human subjects at about 37° C. for about 24 h. It has now been discovered that for individuals who have osteoarthritis, that therapeutically effective concentrations of IL-1ra and TIMPs are achieved by incubating or storing human blood admixed with sodium citrate at a temperature of about 20° C. to about 40° C. for about 3.5 hours to about 6 hours or more.

IL-4, 10, 13, PDGF, TGFβ are included in the content of platelets and-granules and are delivered in the PRP component. IL-4, 10, 13 come from white blood cells. PDGF is produced by platelets and TGFβ is released by platelets and some T cells. Employing the regenerative effect of the mentioned proteins leads to generation of a potent bio-active autologous product. Thus, a combination of fresh-prepared PRP as a source of regenerative biological factors and anti-inflammatory cytokines and growth factors, and the anti-inflammatory component comprising stored autologous serum as a source of IL-1 inhibitor provides a powerful and cost-effective autologous therapeutic agent for treatment of degenerative conditions like osteoarthritis, chronic tendinosis and chronic muscle tears as well as skin inflammatory disorders.

As used herein, “treatment” includes palliative treatment, wherein pain and/or inflammation is reduced in the subject.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word that it modifies.

The described method for producing an autologous composition for the treatment of osteoarthritis, chronic tendinosis and chronic muscle tear as well as skin inflammatory disorders preferably comprises the step of collecting a mammal's autologous physiological fluid, preferably blood by an aseptic technique. Preferably, the mammal is a human. However, the compositions and methods hereof are also suitable for a wide range of veterinary applications, for example for the treatment of horses, dogs and camels.

The site of venipuncture and the surface of the collection tubes may be cleaned with a 2 percent tincture of iodine solution. Before any cleansing of the site is begun, the patient may be asked about any allergy to iodine. Alternatively, the site of venipuncture and the surface of the collection tubes may be cleaned with a solution of 2% chlorhexidine gluconate in 70% isopropyl alcohol solution. The tube covers are cleaned with 70% alcohol solution also to avoid possible contamination before blood collection.

The autologous anti-inflammatory/anti-catabolic composition is preferably prepared by incubating or storing autologous physiological fluid, preferably blood from a mammal, preferably a human admixed with sodium citrate, at room temperature of preferably about 20° C. However, the blood admixed with sodium citrate can be incubated or stored at temperatures of from about 20° C. to about 40° C. with acceptable results.

The blood admixed with sodium citrate is incubated or stored preferably for about 3.5 hours to about 12 hours at about 20° C. for IL-1ra extracellular enrichment and preferably for the production of TIMPs. The blood admixed with sodium citrate is incubated or stored most preferably for about 3.5 hours to about 6 hours at about 20° C. However, the blood admixed with sodium citrate can be stored for longer than 12 hours and at temperatures of from about 20° C. to about 40° C. with acceptable results, as mentioned above.

A therapeutically effective amount of citrate in the form of sodium citrate, is added to preferably a sterile glass tube or a polystyrene tube into which the blood is collected prior to incubation. The sodium citrate provided is preferably a 4% by weight solution of sodium citrate. An example of an acceptable 4% by weight solution of sodium citrate is Anticoagulant Sodium Citrate Solution USP provided by Baxter Corporation under DIN 00060313 issued by Health Canada, containing 4 g of sodium citrate dihydrate per 100 ml of solution. In a particularly preferred embodiment, the incubation can be in sterile glass tubes (Coviden) or polystyrene (BD) vacutainer tubes with no additives. Further provided in an embodiment is the incubation of an autologous physiological fluid, preferably blood, on a rocker platform (24 rpm) or in static conditions. Preferably incubation is carried out in static conditions.

Preferably, the storing of blood is in the presence of 0.64-0.72 mM Ca⁺⁺ to facilitate IL-1ra production. It is possible and advantageous in a particularly preferred embodiment to dilute cultured blood with sterile calcium chloride solution containing 0.64-0.72 mM Ca⁺⁺ in 9:1 proportion by adding the solution using a sterile syringe and needle directly to the tube with blood before the incubation (1 cc of the calcium chloride solution to 9 cc whole blood). An equal part of sterile air may be added to the sterile tubes containing the blood to expose the culture to atmospheric air for increasing IL-1ra production. In a particularly preferred embodiment, the air is passed through a 0.22 μm MillexGP filter using a sterile syringe and needle directly to the tube with the blood before the incubation.

The sodium citrate solution at a concentration of 4% by weight of sodium citrate is admixed with the blood prior to incubation preferably in a ratio of 9.5 parts of whole blood (9.5 cc):0.5 parts of the 4% by weight sodium citrate solution (0.5 cc).

The incubated admixture of blood and sodium citrate is then subjected to centrifugation to separate a supernatant component from the cellular fraction. The supernatant component is the resultant autologous anti-inflammatory/anti-catabolic composition. The centrifugation is carried out according to methods known in the art. Preferably, the centrifugation is carried out for about 10-20 minutes at about 4000-10000 rpm. Most preferably the centrifugation is carried out for 10 minutes at 4000 rpm.

After centrifugation, the supernatant is preferably filtered through a 0.25 μm filter. The supernatant can be combined with the regenerative autologous platelet-rich plasma (PRP) composition immediately or can optionally be divided into aliquots for future processing using a sterile technique. The procedure is carried out in a sterile environment (laminar flow hood with HEPA filters). Preferably, about three cc of the supernatant containing biologically active agents are carefully drawn by sterile syringe and needle. Prolonged storage of IL-1ra containing product can be accomplished by freezing aliquots at about −20° C. and storing for up to 18 months at about −70° C.

The preparation of the regenerative autologous platelet-rich plasma (PRP) composition involves drawing blood into vacutainer tubes. The blood is then mixed with an anticoagulant according to methods known in the art. The preferred anticoagulant of the present disclosure is sodium citrate. Most preferably, the anticoagulant is a 4% by weight sodium citrate solution. Preferably, the anticoagulant is provided in a 9.5 parts of whole blood (9.5 cc):0.5 parts of 4% by weight sodium citrate (0.5 cc) ratio. A person skilled in the art will appreciate that other anticoagulants such as acid citrate dextrose solution and heparin can be used as the anticoagulant in the preparation of the regenerative autologous platelet-rich plasma (PRP) composition.

The blood is then subjected to centrifugation according to methods known in the art, preferably for about 30 seconds, at about 7500 rpm, to isolate the PRP fraction. The PRP fraction obtained as a product of the centrifugation step is the autologous PRP composition. The centrifugation parameters are used in preferred embodiments for the PRP preparation as a part of the final product for the osteoarthritis and chronic tendinosis treatment and skin disorders. The autologous PRP composition is drawn by a sterile syringe and needle under sterile conditions. In a particularly preferred embodiment for the treatment of chronic tear, a leukocyte buffy coat fraction is added to the autologous PRP composition as an additional VEGF source in order to promote new blood vessel development in the affected site. The buffy coat layer and plasma is collected manually by sterile syringe and needle after whole blood centrifugation as set out above or using a commercially available Harvest SmartPrep system.

The autologous PRP composition is optionally activated by filtering autologous PRP composition from the syringe through a small pore filter which is preferably an about 0.25 μm filter and most preferably a 0.22 μm MillexGP filter.

A final product is prepared comprised of a 50/50 combination of anti-inflammatory and regenerative (activated PRP) compositions by mixing the anti-inflammatory/anti-catabolic composition with the platelet rich plasma composition to provide the autologous composition.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

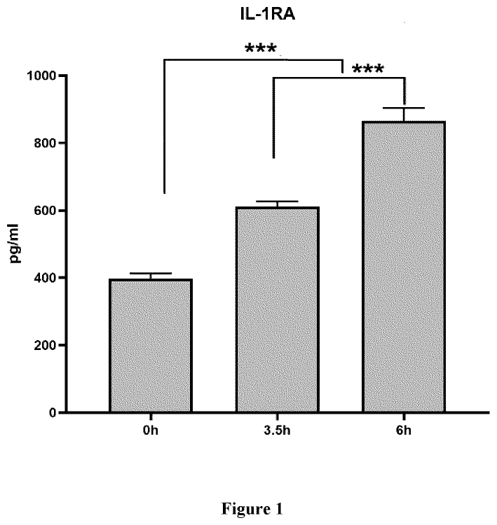

FIG. 1 demonstrates a significant production of IL-1ra in the blood of three human patients diagnosed with bilateral knee osteoarthritis where the blood was drawn from the patients and stored at room temperature of about 20° C. for either 3.5 hours or 6 hours in the presence of sodium citrate. The data shown in FIG. 1 is based on average results from the three patients, the details of which are provided below.

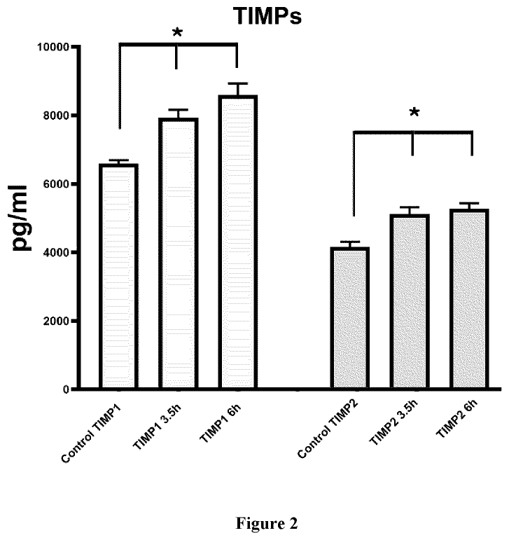

FIG. 2 demonstrates a significant production of TIMP 1 and TIMP 2 in the blood of the three patients diagnosed with bilateral knee osteoarthritis where the blood was stored at room temperature of about 20° C. for either 3.5 hours or 6 hours in the presence of sodium citrate. The data shown in FIG. 2 is based on average results from the three patients, the details of which are provided below.

The results demonstrate a significant production of IL-1ra, TIMP 1 and TIMP 2 in the blood of three human patients diagnosed with bilateral knee osteoarthritis where the blood was stored in the presence of sodium citrate at room temperature of about 20° C. for either 3.5 hours or 6 hours.

This was an unexpected result as blood stored in the absence of sodium citrate had not been previously demonstrated to produce significant levels of IL-1Ra, TIMP 1 and TIMP 2 after 3.5 hours or 6 hours of incubation.

The data shown in FIGS. 1 and 2 was obtained from the patients in cases 1, 2 and 3 from example 2 below.

### Example 2

Each patient was an individual diagnosed with bilateral knee osteoarthritis. For each patients, blood was collected in separate glass and plastic tubes containing sodium citrate. Each glass tube contained about 9.5 parts of whole blood (9.5 cc) and 0.5 parts of a 4% by weight sodium citrate solution (0.5 cc).

The glass tubes containing the patient's blood were stored for either 3.5 h or 6 h at about 20° C. The levels of IL-1ra, TIMP 1 and TIMP 2 in the blood of the patients were then measured. The results for the three patients mentioned above are shown in FIGS. 1 and 2.

The glass tubes and were then centrifuged for 10 min at 4,000 4rpm to isolate the anti-inflammatory component. After centrifugation, the anti-inflammatory component was filtered through a 0.25 μm filter.

The blood collected in the plastic tubes was used to prepare the regenerative PRP component. The plastic tubes were centrifuged immediately at about 7500 rpm and the regenerative component was collected to a sterile syringe. The regenerative component from the syringe was then filtered and thereby activated by passing through a 0.25 μm filter. The regenerative component was then immediately combined with the anti-inflammatory component that had been stored for either 3.5 h or 6 h at about 20° C.

A final autologous composition was obtained comprising a 50/50 combination of the anti-inflammatory and regenerative (activated PRP) components. In each case, the autologous composition was administered to the patient.

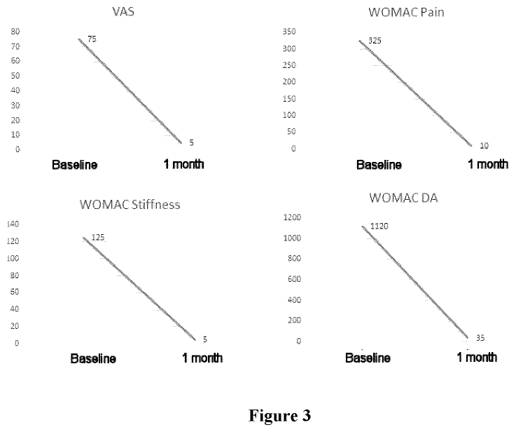

Each patient was assessed with the Western Ontario and McMaster Universities Arthritis Index (WOMAC) questionnaire for assessing pain, stiffness, and physical function in patients with hip and/or knee osteoarthritis. A one-month post-injection preliminary analysis of the WOMAC questionnaire data showed a statistically significant improvement in each of the patients' pain, stiffness and daily activities as shown in FIGS. 3-6. A statistical analysis of Visual Analog Pain Scale (VAS) revealed a significant pain reduction in each of the patients, as also shown in FIGS. 3-6.

Case 1: 31 years

Diagnosis: The patient reported onset of right knee pain. An MRI of the right knee showed osteoarthritis changes in the form of a complex tear of the body and posterior horn medial meniscus, inflamed plica and mid chondromalacia, 5 cm Bakers cyst.

Treatment: Right knee autologous composition injections into knees×1. The blood was stored for 6 hours at room temperature.

Result: As shown in FIG. 3, at a one month follow up after injection, the patient reported significant improvement in terms of pain reduction, stiffness reduction, daily activities and a VAS score revealing a significant pain reduction after one month. The results show a strong improvement in WOMAC scores. The patient was able to resume physical activity.

Case 2: 59 years

Diagnosis: The patient reported onset of left knee pain. An MRI of the left knee showed osteoarthritis changes: the presence of an associated horizontal cleavage tear, degeneration of the lateral meniscus.

Treatment: Left knee autologous composition injections into knees×1. The glass tubes containing the patient blood for the preparation of the anti-inflammatory component were left stored for 6 hours at room temperature.

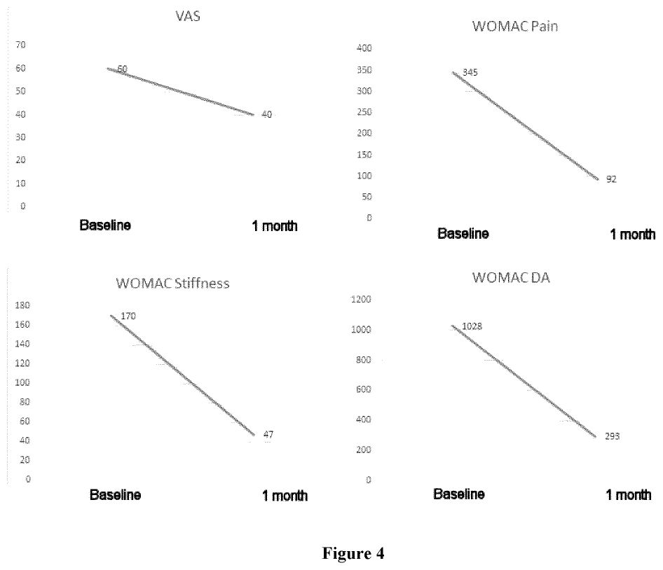

Result: As shown in FIG. 4, at a one month follow up after injection, the patient reported significant improvement, strong pain reduction, strong improvement in WOMAC and VAS scores.

Case 3: 62 years

Diagnosis: The patient reported onset of left knee pain. An MRI of the left knee showed osteoarthritis changes: complex tear involving a body and posterior horn of the medial meniscus with degeneration, degenerative thinning of the articular hyaline cartilage overlying femoral condyles and medial tibial plateau.

Treatment: Left knee autologous composition injections into knees×1. The glass tubes containing the subject's blood for the preparation of the anti-inflammatory component were stored for 3.5 hours at room temperature.

Result: As shown in FIG. 5, at one month follow up after injection, the patient reported significant therapeutic effect, strong pain reduction, strong improvement in WOMAC and VAS scores.

Case 4: 70 years

Diagnosis: The patient reported onset right knee pain. An MRI of the right knee showed osteoarthritis changes: 0.5×0.4 cm mixed partial and full-thickness cartilage defect involving the weightbearing surface of the lateral femoral condyle with 10 mm cluster of subchondral cyst: moderate tricompartmental osteoarthritis.

Treatment: Right knee autologous composition injections×1. The glass tubes containing the subject's blood for the preparation of the anti-inflammatory component were stored for 3.5 hours at room temperature.

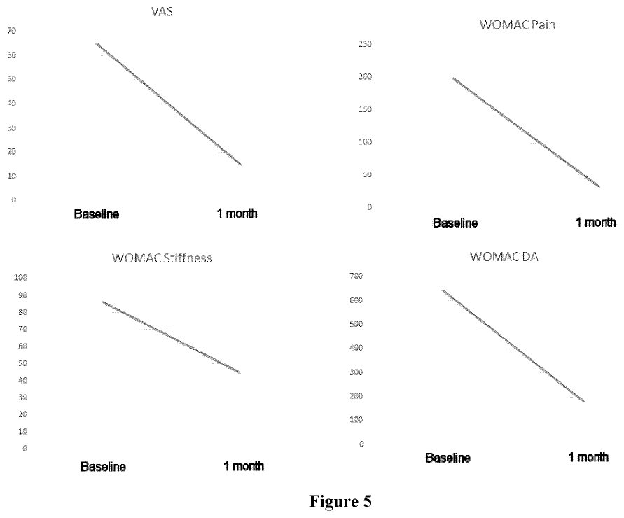

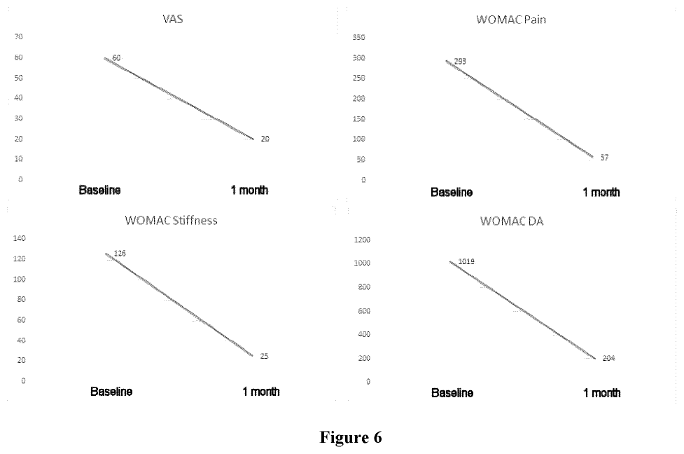

Result: As shown in the graphs shown in FIG. 6, at one month follow up after injection, the patient reported significant therapeutic effect, strong pain reduction, strong improvement in WOMAC and VAS scores.

### Example 3

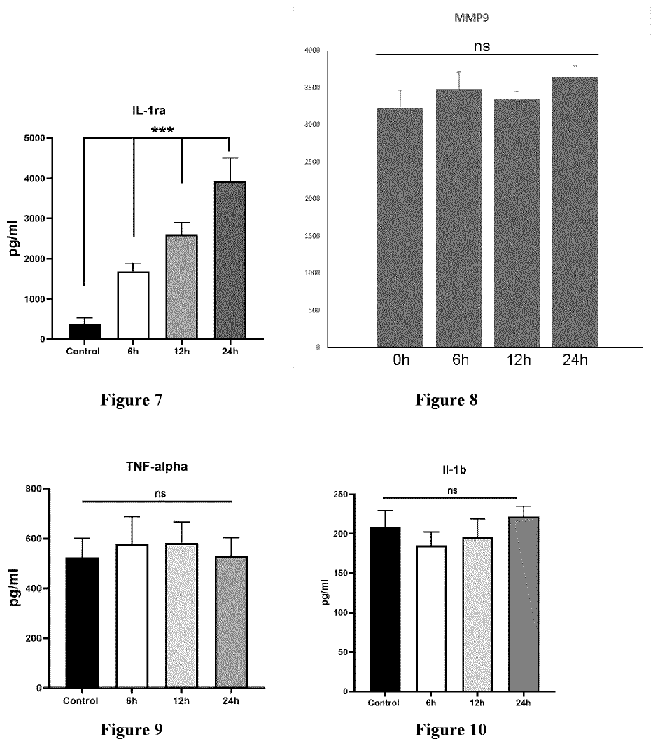

Blood was drawn from 12 healthy subjects and then was mixed with a 4% by weight sodium citrate solution. The blood in admixture with a 4% by weight sodium citrate solution was stored at about 20° C. The blood to sodium citrate ratio was 9.5 parts of whole blood (9.5 cc):0.5 parts of 4% by weight sodium citrate solution (0.5 cc). Measurements of levels of IL-1ra, MMP9, TNFα, and ILβ were taken at 0 hours as a control, 6 hours, 12 hours and 24 hours. The results are shown in FIGS. 7-10.

FIG. 7 shows an average level of IL-1ra among the 12 subjects at 0 hours as a control, 6 hours, 12 hours and 24 hours. The results as shown in FIG. 7 show a statistically significant increase in the level of IL-1ra after 6 hours and 12 hours of incubation. This is statistically significant according to a one-way analysis of variance analysis.

FIG. 8 shows an average level of MMP9 among the 12 subjects at 0 hours as a control, 6 hours, 12 hours and 24 hours. The results as shown in FIG. 8 show no increase in the level of MMP9 after incubation.

FIG. 9 shows an average level of TNFα among the 12 subjects at 0 hours as a control, 6 hours, 12 hours and 24 hours. The results as shown in FIG. 9 show no increase in the level of TNFα after incubation.

FIG. 10 shows an average level of ILβ among the 12 subjects at 0 hours as a control, 6 hours, 12 hours and 24 hours. The results as shown in FIG. 10 show no increase in the level of ILβ after incubation.

### Example 4

Twenty-two patients were treated with the autologous composition of the present disclosure. Each patient was an individual diagnosed with bilateral knee osteoarthritis. For each patient, blood was collected in separate glass and plastic tubes containing sodium citrate. Each glass tube contained about 9.5 parts of whole blood (9.5 cc) and 0.5 parts of 4% by weight sodium citrate solution (0.5 cc).

The glass tubes containing the patients' blood in admixture with sodium citrate were stored for 6 hours at about 20° C. The glass tubes were then centrifuged for 10 min at 4,000 rpm to isolate the anti-inflammatory component. After centrifugation, the anti-inflammatory component was filtered through a 0.25 μm filter.

The blood collected in the plastic tubes was used to prepare the regenerative PRP component. The plastic tubes were centrifuged immediately at about 7500 rpm and the regenerative component was collected to a sterile syringe. The regenerative component from the syringe was then filtered and thereby activated by passing through a 0.25 μm filter. The regenerative component was then immediately combined with the anti-inflammatory component that had been stored for 6 hours at about 20° C.

For each patient, a final autologous composition was obtained comprising a 50/50 combination of the anti-inflammatory and regenerative (activated PRP) components. In each case, the autologous composition was administered to the patient.

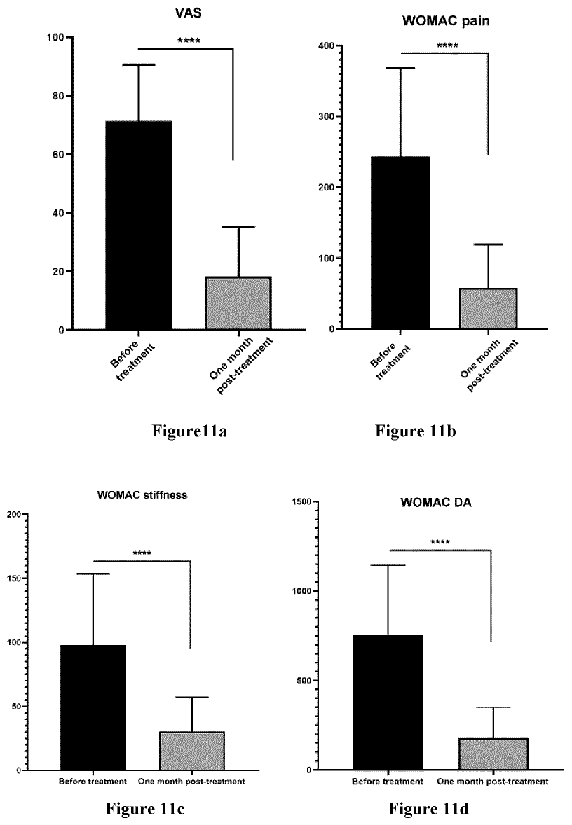

The twenty-two patients were assessed with the Western Ontario and McMaster Universities Arthritis Index (WOMAC) questionnaire for assessing pain, stiffness, and physical function in patients with hip and/or knee osteoarthritis. A one-month post-injection preliminary analysis of the WOMAC questionnaire data showed a statistically significant improvement in the patients' pain, stiffness and daily activities in patients treated with the autologous composition as shown in FIGS. 11b, 11c, and 11d respectively. A statistical analysis of Visual Analog Pain Scale (VAS) revealed a significant pain reduction in the patients, as shown in FIG. 11a.

Although the invention has been described with reference to illustrative embodiments, it is to be understood that the invention is not limited to these precise embodiments. Numerous modifications, variations, and adaptations may be made to the particular embodiments of the invention described above without departing from the scope of the invention. The scope of the claims should not be limited by the preferred embodiments set forth in the examples, but should be given the broadest interpretation consistent with the description as a whole.

1-6. (canceled)

7. A method of treating a mammal suffering from damaged and/or injured connective tissues, chronic tendinosis, chronic muscle tears and/or chronic degenerative joint conditions and skin inflammatory disorders with an autologous anti-inflammatory/anti-catabolic composition, the method comprising the following steps:
admixing blood from the mammal with a quantity of sodium citrate to form an admixture;
incubating the admixture at a temperature of from about 20° C. to about 40° C. for at least about 3.5 hours to about 12 hours;
centrifuging the incubated admixture to separate the blood into a supernatant component and a cellular fraction;
collecting the supernatant component to provide the autologous anti-inflammatory/anti-catabolic composition; and
administering the autologous anti-inflammatory/anti-catabolic composition to the mammal.

8. The method according to claim 7, wherein the mammal is a human.

9. The method according to claim 7, wherein the admixture is incubated for about 3.5 hours.

10. The method according to claim 7, wherein the admixture is incubated for about 6 hours.

11. The method according to claim 7 wherein the sodium citrate is a 4% by weight sodium citrate solution.

12. The method according to claim 11, wherein the step of admixing blood from the mammal with a quantity of sodium citrate to from an admixture comprises providing a ratio of 9.5 parts of whole blood to 0.5 parts of the 4% by weight sodium citrate solution.

13. The method according to claim 11 wherein 9.5 cc of blood is mixed with 0.5 cc of the 4% by weight sodium citrate solution.

14. A method of producing an autologous composition useful in the treatment of a mammal suffering from damaged and/or injured connective tissues, chronic tendinosis, chronic muscle tears, chronic degenerative joint conditions, and/or skin inflammatory disorders, the method comprising the following steps:
preparing an anti-inflammatory/anti-catabolic component of the autologous composition comprising IL-1ra and TIMPs, said step of preparing the anti-inflammatory/anti-catabolic component comprising the following steps:
admixing blood from the mammal with a quantity of sodium citrate to form an admixture;
incubating the admixture at a temperature of from about 20° C. to about 40° C. for at least about 3.5 hours to about 12 hours;
centrifuging the incubated admixture to separate the blood into a supernatant component and a cellular fraction;
collecting the supernatant component of the anti-inflammatory/anti-catabolic component; preparing a regenerative component of the autologous composition comprising the following steps: mixing blood from the mammal with a quantity of an anticoagulant;
centrifuging the blood to separate a platelet rich plasma component therefrom;
collecting the platelet rich plasma component; and
mixing the supernatant component of the anti-inflammatory/anti-catabolic component with the platelet rich plasma component to provide the autologous composition.

15. The method according to claim 14, wherein the admixture is incubated for about 3.5 hours.

16. The method according to claim 14, wherein the admixture is incubated for about 6 hours.

17. The method according to claim 14 wherein the sodium citrate is a 4% by weight sodium citrate solution.

18. The method according to claim 17, wherein the step of admixing blood from the mammal with a quantity of sodium citrate to from an admixture comprises providing a ratio of 9.5 parts of whole blood to 0.5 parts of the 4% by weight sodium citrate solution.

19. The method according to claim 17 wherein 9.5 cc of blood is mixed with 0.5 cc of the 4% by weight sodium citrate solution.

20. The method according to claim 14 wherein the anticoagulant is an about 4% by weight sodium citrate solution.

21. A method of treating a mammal suffering from damaged and/or injured connective tissues, chronic tendinosis, chronic muscle tears and/or chronic degenerative joint conditions and skin inflammatory disorders, the method comprising the following steps:
preparing an anti-inflammatory/anti-catabolic component an autologous composition comprising IL-1ra and TIMPs, said step of preparing the anti-inflammatory/anti-catabolic component comprising the following steps:
admixing blood from the mammal with a quantity of sodium citrate to form an admixture;
incubating the admixture at a temperature of from about 20° C. to about 40° C. for at least about 3.5 hours to about 12 hours;
centrifuging the incubated admixture to separate the blood into a supernatant component and a cellular fraction; and
collecting the supernatant component of the anti-inflammatory/ anti-catabolic component;
preparing a regenerative component of the autologous composition comprising the following steps:
mixing blood from the mammal with a quantity of an anticoagulant;
centrifuging the blood to separate a platelet rich plasma component therefrom;
collecting the platelet rich plasma component;
mixing the supernatant component with the platelet rich plasma component to provide the autologous composition; and
administering the autologous composition to the mammal.

22. The method according to claim 21, wherein the admixture is incubated for about 3.5 hours.

23. The method according to claim 21, wherein the admixture is incubated for about 6 hours.

24. The method according to claim 21 wherein the sodium citrate is a 4% by weight sodium citrate solution.

25. The method according to claim 24, wherein the step of admixing blood from the mammal with a quantity of sodium citrate to from an admixture comprises providing a ratio of 9.5 parts of whole blood to 0.5 parts of the 4% by weight sodium citrate solution.

26. The method according to claim 24 wherein 9.5 cc of blood is mixed with 0.5 cc of the 4% by weight sodium citrate solution.

27. (canceled)
